SEQUENCE LISTING (29 sequences)

SEQ ID NO: 1 - DNA, 20 bp, Artificial
  A synthetic oligonucleotide bound to a microarray substrate to serve as a proble molecule
aaaaaaaaaaaaaaaatctc

SEQ ID NO: 2 - DNA, 21 bp, Artificial
  A synthetic oligonucleotide bound to the surface of a microarray to serve as a probe molecule
aaaaaaaaaaaaaaaatctcc

SEQ ID NO: 3 - DNA, 22 bp, Artificial
  A synthetic oligonucleotide bound to the surface of a microarray to serve as a probe molecule.
aaaaaaaaaaaaaaaatctccc

SEQ ID NO: 4 - DNA, 23 bp, Artificial
  A synthetic oligonucleotide bound to the surface of a microarray to serve as a probe molecule.
aaaaaaaaaaaaaaaatctccca

SEQ ID NO: 5 - DNA, 23 bp, Artificial
  A synthetic oligonucleotide bound to the surface of a microarray to serve as a probe molecule.
aaaaaaaaaaaaaaaaaatctcc

SEQ ID NO: 6 - DNA, 24 bp, Artificial
  A synthetic oligonucleotide bound to the surface of a microarray to serve as a probe molecule.
aaaaaaaaaaaaaaaatctcccaa

SEQ ID NO: 7 - DNA, 24 bp, Artificial
  A synthetic oligonucleotide bound to the surface of a microarray to serve as a probe molecule.
aaaaaaaaaaaaaaaaaatctccc

SEQ ID NO: 8 - DNA, 25 bp, Artificial
  A synthetic oligonucleotide bound to the surface of a microarray to serve as a probe molecule.
aaaaaaaaaaaaaaaatctcccaaa

SEQ ID NO: 9 - DNA, 25 bp, Artificial
  A synthetic oligonucleotide bound to the surface of a microarray to serve as a probe molecule.
aaaaaaaaaaaaaaaaaatctccca

SEQ ID NO: 10 - DNA, 26 bp, Artificial
  A synthetic oligonucleotide bound to the surface of a microarray to serve as a probe molecule.
aaaaaaaaaaaaaaaatctcccaaaa

SEQ ID NO: 11 - DNA, 26 bp, Artificial
  A synthetic oligonucleotide bound to the surface of a microarray to serve as a probe molecule.
aaaaaaaaaaaaaaaaaatctcccaa

SEQ ID NO: 12 - DNA, 27 bp, Artificial
  A synthetic oligonucleotide bound to the surface of a microarray to serve as a probe molecule.
aaaaaaaaaaaaaaaaaatctcccaaa

SEQ ID NO: 13 - DNA, 27 bp, Artificial
  A synthetic oligonucleotide bound to the surface of a microarray to serve as a probe molecule.
aaaaaaaaaaaaaaaatctcccaaaaa

SEQ ID NO: 14 - DNA, 28 bp, Artificial
  A synthetic oligonucleotide bound to the surface of a microarray to serve as a probe molecule.
aaaaaaaaaaaaaaaaaatctcccaaaa

SEQ ID NO: 15 - DNA, 28 bp, Artificial
  A synthetic oligonucleotide bound to the surface of a microarray to serve as a probe molecule.
aaaaaaaaaaaaaaaaaatctcccaaaa

SEQ ID NO: 16 - DNA, 28 bp, Artificial
  A synthetic oligonucleotide bound to the surface of a microarray to serve as a probe molecule.
aaaaaaaaaaaaaaaatctcccaaaaaa

SEQ ID NO: 17 - DNA, 29 bp, Artificial
  A synthetic oligonucleotide bound to the surface of a microarray to serve as a probe molecule.
aaaaaaaaaaaaaaaaaatctcccaaaaa

SEQ ID NO: 18 - DNA, 29 bp, Artificial
  A synthetic oligonucleotide bound to the surface of a microarray to serve as a probe molecule.
aaaaaaaaaaaaaaaatctcccaaaaaaa

SEQ ID NO: 19 - DNA, 30 bp, Artificial
  A synthetic oligonucleotide bound to the surface of a microarray to serve as a probe molecule.
aaaaaaaaaaaaaaaaaatctcccaaaaaa

SEQ ID NO: 20 - DNA, 30 bp, Artificial
  A synthetic oligonucleotide bound to the surface of a microarray to serve as a probe molecule.
aaaaaaaaaaaaaaaatctcccaaaaaaaa

SEQ ID NO: 21 - DNA, 31 bp, Artificial
  A synthetic oligonucleotide bound to the surface of a microarray to serve as a probe molecule.
aaaaaaaaaaaaaaaaaatctcccaaaaaaa

SEQ ID NO: 22 - DNA, 31 bp, Artificial
  A synthetic oligonucleotide bound to the surface of a microarray to serve as a probe molecule.
aaaaaaaaaaaaaaaatctcccaaaaaaaaa

SEQ ID NO: 23 - DNA, 32 bp, Artificial
  A synthetic oligonucleotide bound to the surface of a microarray to serve as a probe molecule.
aaaaaaaaaaaaaaaaaatctcccaaaaaaaa

SEQ ID NO: 24 - DNA, 32 bp, Artificial
  A synthetic oligonucleotide bound to the surface of a microarray to serve as a probe molecule.
aaaaaaaaaaaaaaaatctcccaaaaaaaaaa

SEQ ID NO: 25 - DNA, 33 bp, Artificial
  A synthetic oligonucleotide bound to the surface of a microarray to serve as a probe molecule.
aaaaaaaaaaaaaaaaaatctcccaaaaaaaaa

SEQ ID NO: 26 - DNA, 33 bp, Artificial
  A synthetic oligonucleotide bound to the surface of a microarray to serve as a probe molecule.
aaaaaaaaaaaaaaaatctcccaaaaaaaaaaa

SEQ ID NO: 27 - DNA, 34 bp, Artificial
  A synthetic oligonucleotide bound to the surface of a microarray to serve as a probe molecule.
aaaaaaaaaaaaaaaaaatctcccaaaaaaaaaa

SEQ ID NO: 28 - DNA, 35 bp, Artificial
  A synthetic oligonucleotide bound to the surface of a microarray to serve as a probe molecule.
aaaaaaaaaaaaaaaaaatctcccaaaaaaaaaaa

SEQ ID NO: 29 - DNA, 60 bp, Artificial
  A synthetic oligonucleotide bound to the surface of a microarray to serve as a probe molecule.
ttgattcttttttaataaactactctttgatttaaaaaaaaaaaaaaaaaaaaaaaaaaa

(19) United States
(12) Patent Application Publication
(10) Pub. No.: US 2009/0068648 A1
(43) Pub. Date: Mar. 12, 2009

(54) Method and system for determining a quality metric for comparative genomic hybridization experimental results

(76) Inventors: Zohar Yakhini, Ramat Hasharon (IL); Amir Bon-Dor, Bellevue, WA (US); Anya Tsalenko, Chicago, IL (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 11/580,345

(22) Filed: Oct. 13, 2006

(51) Int. Cl. C12Q 1/68

(52) U.S. Cl. 435/6

(57) ABSTRACT

Various embodiments of the present invention determine various quality metrics that reflect the quality of two or more identically-executed or similar array-based comparative-genomic-hybridization (“aCGH”) experiments. In certain embodiments of the present invention, a pairwise quality metric is generated for each possible pair of aCGH experimental results within a set of aCGH experimental results. The pairwise quality metrics may be summed and optionally normalized to produce an overall quality metric for the set of aCGH experimental results. Various pairwise quality metrics can be used in different embodiments of the present invention, including pairwise quality metrics based on measures of aberration overlap.

### TECHNICAL FIELD OF THE INVENTION

The present invention is related to analysis of comparative genomic hybridization data, quality control of array-based experiments and experimental results, and, in particular, to methods and systems for determining various quality metrics for multiple identically-executed or similar comparative-genomic-hybridization experiments.

### BACKGROUND OF THE INVENTION

Significant research efforts have been devoted to elucidate the causes and cellular mechanisms responsible for transformation of normal cells to precancerous and cancerous states and for the growth of, and metastasis of, cancerous tissues. Enormous strides have been made in understanding various causes and cellular mechanisms of cancer, and this detailed understanding is currently providing new and useful approaches for preventing, detecting, and treating cancer.

There are myriad different types of causative events and agents associated with the development of cancer, and there are many different types of cancer and many different patterns of cancer development for each of the many different types of cancer. Although initial hopes and strategies for treating cancer were predicated on finding one or a few basic, underlying causes and mechanisms for cancer, researchers have, over time, recognized that what they initially described generally as “cancer” appears to, in fact, be a very large number of different diseases. Nonetheless, there do appear to be certain common cellular phenomena associated with the various diseases described by the term “cancer.” One common phenomenon, evident in many different types of cancer, is the onset of genetic instability in precancerous tissues, and progressive genomic instability as cancerous tissues develop. While there are many different types and manifestations of genomic instability, a change in the number of copies of particular DNA subsequences within chromosomes and changes in the number of copies of entire chromosomes within a cancerous cell may be a fundamental indication of genomic instability. Although cancer is one important pathology correlated with genomic instability, changes in gene copies within individuals, or relative changes in gene copies between related individuals, may also be causally related to, correlated with, or indicative of other types of pathologies and conditions, for which techniques to detect gene-copy changes may serve as useful diagnostic, treatment development, and treatment monitoring aids.

Various techniques have been developed to detect and at least partially quantify amplification and deletion of chromosomal DNA subsequences in cancerous cells. One technique is referred to as “comparative genomic hybridization.” Comparative genomic hybridization (“CGH”) can offer striking, visual indications of chromosomal-DNA-subsequence amplification and deletion, in certain cases, but, like many biological and biochemical analysis techniques, is subject to significant noise and sample variation, leading to problems in quantitative analysis of CGH data. Array-based comparative genomic hybridization (“aCGH”) has been relatively recently developed to provide a higher resolution, highly quantitative comparative-genomic-hybridization technique. In addition to studying cancer, aCGH and CGH techniques can be used to study evolutionary genetics, developmental disorders, antibiotic resistance, and a host of other genetically-driven phenomena. As with all experimental techniques, it is important for researchers and clinicians to be able to ascertain the quality of aCGH experimental results and use quantitative measures of the quality in drawing conclusions from aCGH data. Researchers and developers of aCGH techniques and equipment have recognized the need for reliable methods and systems for evaluating the quality of aCGH-derived experimental data.

### SUMMARY OF THE INVENTION

Various embodiments of the present invention determine various quality metrics that reflect the quality of two or more identically-executed or similar array-based comparative-genomic-hybridization (“aCGH”) experiments. In certain embodiments of the present invention, a pairwise quality metric is generated for each possible pair of aCGH experimental results within a set of aCGH experimental results. The pairwise quality metrics may be summed and optionally normalized to produce an overall quality metric for the set of aCGH experimental results. Various pairwise quality metrics can be used in different embodiments of the present invention, including pairwise quality metrics based on measures of aberration overlap.

### BRIEF DESCRIPTION OF THE DRAWINGS

FIG. 1 shows the chemical structure of a small, four-subunit, single-chain oligonucleotide.

FIG. 2 shows a symbolic representation of a short stretch of double-stranded DNA.

FIG. 3 illustrates construction of a protein based on the information encoded in a gene.

FIG. 4 shows a hypothetical set of chromosomes for a very simple, hypothetical organism.

FIG. 5 shows examples of gene deletion and gene amplification in the context of the hypothetical genome shown in FIG. 4.

FIGS. 6-7 illustrate detection of gene amplification by CGH.

FIGS. 8-9 illustrate detection of gene deletion by CGH.

FIGS. 10-12 illustrate microarray-based CGH.

FIG. 13 illustrates one method for identifying and ranking intervals and removing redundancies from lists of intervals identified as probable deletions or amplifications.

FIGS. 14A-B illustrate two hypothetical aCGH experimental results.

FIG. 15 shows an alternative graphical representation of the two experimental results E₁ and E₂.

FIG. 16 illustrates calculation, according to a method embodiment of the present invention, of an interval-overlap metric O_{i,j} based on two aberrant intervals i and j representing either two amplifications or two deletions within two different experiments results E₁ and E₂.

FIGS. 17A-L illustrate computation of an overall pairwise overlap metric O(E₁, E₂) for the experimental results E₁ and E₂ shown in FIGS. 14A-B, according to a first described method embodiment of the present invention.

FIG. 18 illustrates computation of the alternative interval-overlap metric Oᵢ′ according to a method embodiment of the present invention.

FIGS. 19 and 20 are control-flow diagrams representing a quality-metric calculation for a set of k experimental results according to embodiments of the present invention.

### DETAILED DESCRIPTION OF THE INVENTION

Embodiments of the present invention are directed to methods and systems for evaluating the quality of multiple aCGH-derived experimental results. In a first subsection, below, a discussion of array-based comparative genomic hybridization methods and interval-based aberration-calling methods for analyzing aCGH data sets is provided. In a second subsection, embodiments of the present invention are discussed.

### Array-Based Comparative Genomic Hybridization and Interval-Based aCGH Data Analysis

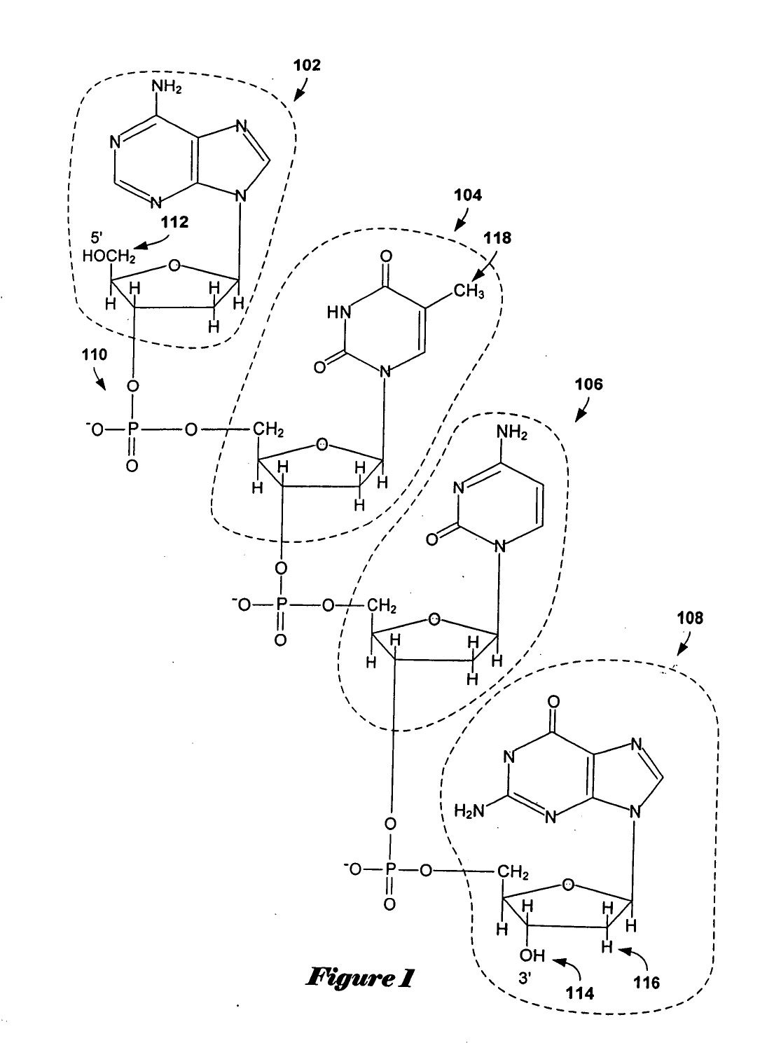

Prominent information-containing biopolymers include deoxyribonucleic acid (“DNA”), ribonucleic acid (“RNA”), including messenger RNA (“mRNA”), and proteins. FIG. 1 shows the chemical structure of a small, four-subunit, single-chain oligonucleotide, or short DNA polymer. The oligonucleotide shown in FIG. 1 includes four subunits: (1) deoxyadenosine 102, abbreviated “A”; (2) deoxythymidine 104, abbreviated “T”; (3) deoxycytodine 106, abbreviated “C”; and (4) deoxyguanosine 108, abbreviated “G.” Each subunit 102, 104, 106, and 108 is generically referred to as a “deoxyribonucleotide,” and consists of a purine, in the case of A and G, or pyrimidine, in the case of C and T, covalently linked to a deoxyribose. The deoxyribonucleotide subunits are linked together by phosphate bridges, such as phosphate 110. The oligonucleotide shown in FIG. 1, and all DNA polymers, is asymmetric, having a 5′ end 112 and a 3′ end 114, each end comprising a chemically active hydroxyl group. RNA is similar, in structure, to DNA, with the exception that the ribose components of the ribonucleotides in RNA have a 2′ hydroxyl instead of a 2′ hydrogen atom, such as 2′ hydrogen atom 116 in FIG. 1, and include the ribonucleotide uridine, similar to thymidine but lacking the methyl group 118, instead of a ribonucleotide analog to deoxythymidine. The RNA subunits are abbreviated A, U, C, and G.

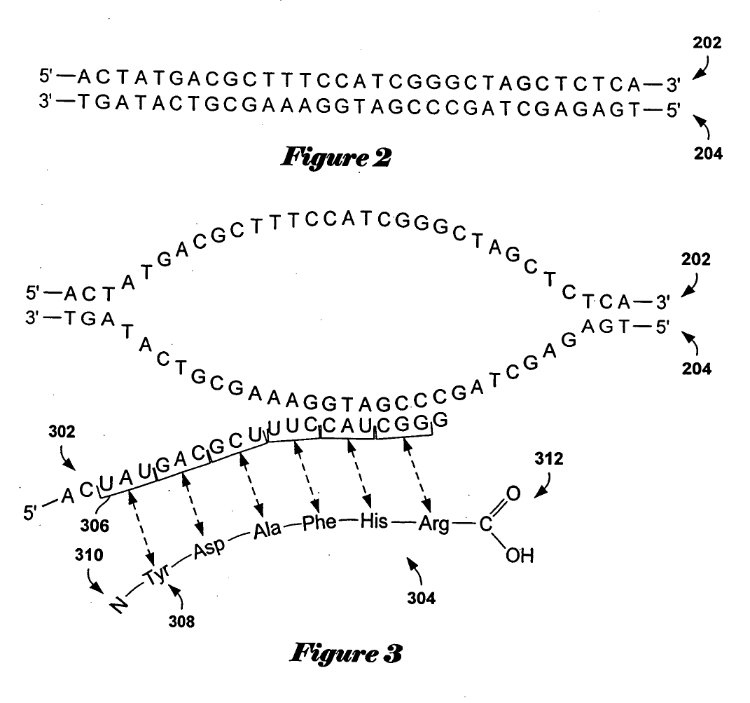

In cells, DNA is generally present in double-stranded form, in the familiar DNA-double-helix form. FIG. 2 shows a symbolic representation of a short stretch of double-stranded DNA. The first strand 202 is written as a sequence of deoxyribonucleotide abbreviations in the 5′ to 3′ direction and the complementary strand 204 is symbolically written in 3′ to 5′ direction. Each deoxyribonucleotide subunit in the first strand 202 is paired with a complementary deoxyribonucleotide subunit in the second strand 204. In general, a G in one strand is paired with a C in a complementary strand, and an A in one strand is paired with a T in a complementary strand. One strand can be thought of as a positive image, and the opposite, complementary strand can be thought of as a negative image, of the same information encoded in the sequence of deoxyribonucleotide subunits.

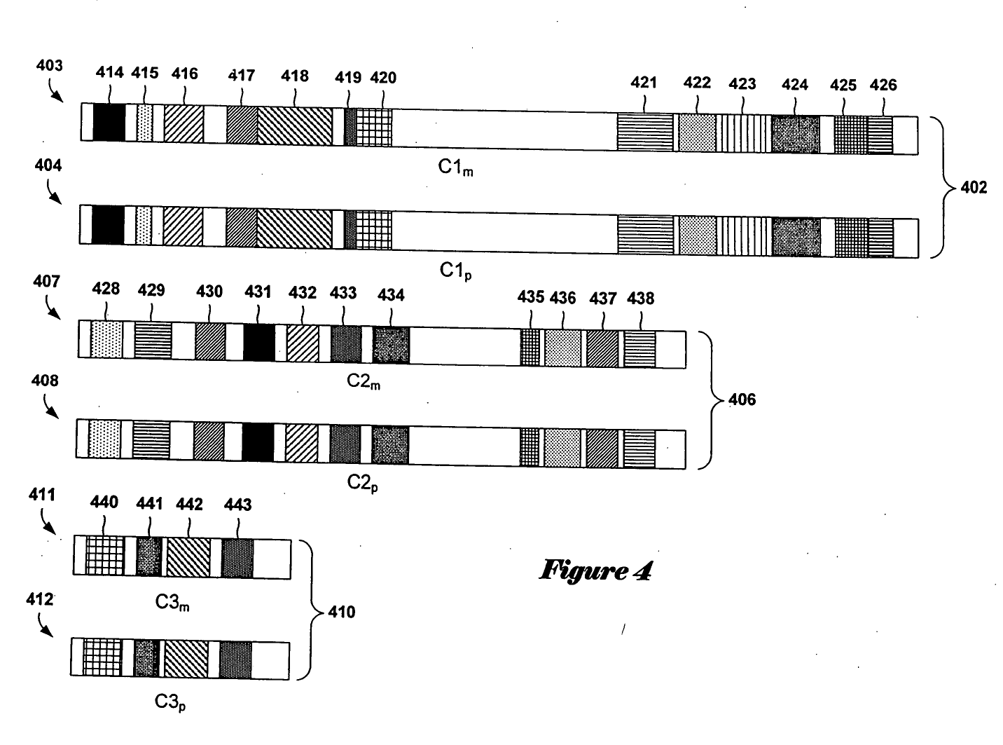

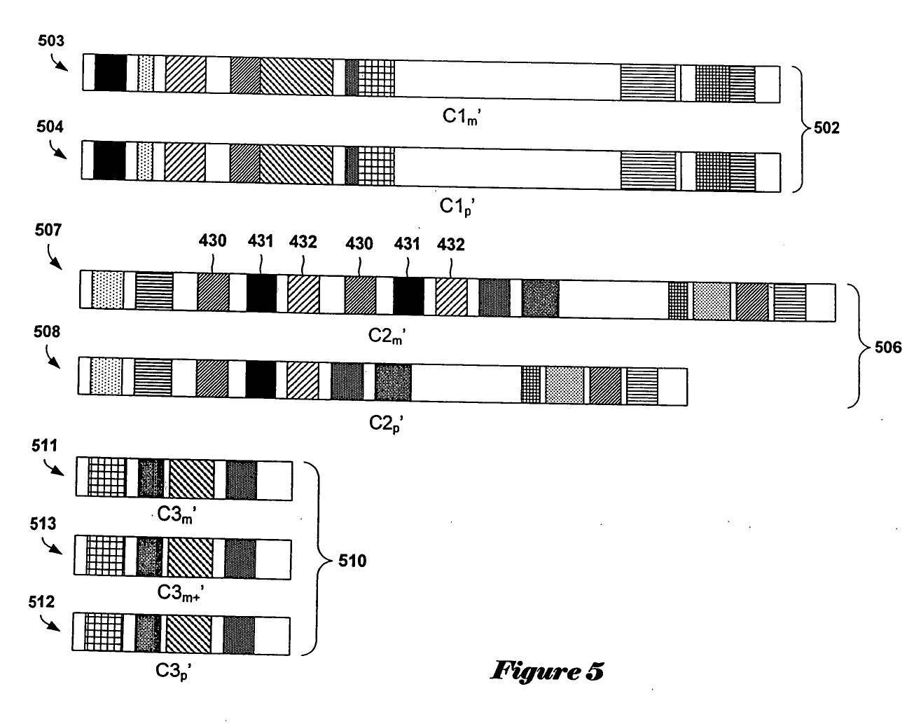

A gene is a subsequence of deoxyribonucleotide subunits within one strand of a double-stranded DNA polymer. One type of gene can be thought of as an encoding that specifies, or a template for, construction of a particular protein. FIG. 3 illustrates construction of a protein based on the information encoded in a gene. In a cell, a gene is first transcribed into single-stranded mRNA. In FIG. 3, the double-stranded DNA polymer composed of strands 202 and 204 has been locally unwound to provide access to strand 204 for transcription machinery that synthesizes a single-stranded mRNA 302 complementary to the gene-containing DNA strand. The single-stranded mRNA is subsequently translated by the cell into a protein polymer 304, with each three-ribonucleotide codon, such as codon 306, of the mRNA specifying a particular amino acid subunit of the protein polymer 304. For example, in FIG. 3, the codon “UAU” 306 specifies a tyrosine amino-acid subunit 308. Like DNA and RNA, a protein is also asymmetrical, having an N-terminal end 310 and a carboxylic acid end 312. Other types of genes include genomic subsequences that are transcribed to various types of RNA molecules, including catalytic RNAs, iRNAs, siRNAs, rRNAs, and other types of RNAs that serve a variety of functions in cells, but that are not translated into proteins. Furthermore, additional genomic sequences serve as promoters and regulatory sequences that control the rate of protein-encoding-gene expression. Although functions have not, as yet, been assigned to many genomic subsequences, there is reason to believe that many of these genomic sequences are functional. For the purpose of the current discussion, a gene can be considered to be any genomic subsequence.

In eukaryotic organisms, including humans, each cell contains a number of extremely long, DNA-double-strand polymers called chromosomes. Each chromosome can be thought of, abstractly, as a very long deoxyribonucleotide sequence. Each chromosome contains hundreds to thousands of subsequences, many subsequences corresponding to genes. The exact correspondence between a particular subsequence identified as a gene, in the case of protein-encoding genes, and the protein or RNA encoded by the gene can be somewhat complicated, for reasons outside the scope of the present invention. However, for the purposes of describing embodiments of the present invention, a chromosome may be thought of as a linear DNA sequence of contiguous deoxyribonucleotide subunits that can be viewed as a linear sequence of DNA subsequences. In certain cases, the subsequences are genes, each gene specifying a particular protein or RNA. Amplification and deletion of any DNA subsequence or group of DNA subsequences can be detected by comparative genomic hybridization, regardless of whether or not the DNA subsequences correspond to protein-sequence-specifying genes, to DNA subsequences specifying various types of RNAs, or to other regions with defined biological roles. The term “gene” is used in the following as a notational convenience, and should be understood as simply an example of a “biopolymer subsequence.” Similarly, although the described embodiments are directed to analyzing DNA chromosomal subsequences extracted from diseased tissues for amplification and deletion with respect to control tissues, the sequences of any information-containing biopolymer are analyzable by methods of the present invention. Therefore, the term “chromosome,” and related terms, are used in the following as a notational convenience, and should be understood as an example of a biopolymer or biopolymer sequence. In summary, a genome, for the purposes of describing the present invention, is a set of sequences. Genes are considered to be subsequences of these sequences. Comparative genomic hybridization techniques can be used to determine changes in copy number of any set of genes of any one or more chromosomes in a genome.

FIG. 4 shows a hypothetical set of chromosomes for a very simple, hypothetical organism. The hypothetical organism includes three pairs of chromosomes 402, 406, and 410. Each chromosome in a pair of chromosomes is similar, generally having identical genes at identical positions along the lines of the chromosome. In FIG. 4, each gene is represented as a subsection of the chromosome. For example, in the first chromosome 403 of the first chromosome pair 402, 13 genes are shown, 414-426.

As shown in FIG. 4, the second chromosome 404 of the first pair of chromosomes 402 includes the same genes, at the same positions, as the first chromosome. Each chromosome of the second pair of chromosomes 406 includes eleven genes 428-438, and each chromosome of the third pair of chromosomes 410 includes four genes 440-443. In a real organism, there are generally many more chromosome pairs, and each chromosome includes many more genes. However, the simplified, hypothetical genome shown in FIG. 4 is suitable for describing embodiments of the present invention. Note that, in each chromosome pair, one chromosome is originally obtained from the mother of the organism, and the other chromosome is originally obtained from the father of the organism. Thus, the chromosomes of the first chromosome pair 402 are referred to as chromosome “C1ₘ” and “C1ₚ.” While, in general, each chromosome of a chromosome pair has the same genes positioned at the same location along the length of the chromosome, the genes inherited from one parent may differ slightly from the genes inherited from the other parent. Different versions of a gene are referred to as alleles. Common differences include single-deoxyribonucleotide-subunit substitutions at various positions within the DNA subsequence corresponding to a gene. Less frequent differences include translocations of genes to different positions within a chromosome or to a different chromosome, a different number of repeated copies of a gene, and other more substantial differences.

Although differences between genes and mutations of genes may be important in the predisposition of cells to various types of cancer, and related to cellular mechanisms responsible for cell transformation, cause-and-effect relationships between different forms of genes and pathological conditions are often difficult to elucidate and prove, and are very often indirect. However, other genomic abnormalities are more easily associated with pre-cancerous and cancerous tissues. Two such prominent types of genomic aberrations include gene amplification and gene deletion. FIG. 5 shows examples of gene deletion and gene amplification in the context of the hypothetical genome shown in FIG. 4. First, both chromosomes C1ₘ′ 503 and chromosome C1ₚ′ 504 of the variant, or abnormal, first chromosome pair 502 are shorter than the corresponding wild-type chromosomes C1ₘ and C1ₚ in the first pair of chromosomes 402 shown in FIG. 4. This shortening is due to deletion of genes 422, 423, and 424, present in the wild-type chromosomes 403 and 404, but absent in the variant chromosomes 503 and 504. This is an example of a double, or homozygous-gene-deletion. Small scale variations of DNA copy numbers can also exist in normal cells. These can have phenotypic implications, and can also be measured by CGH methods and analyzed by the methods of the present invention.

Generally, deletion of multiple, contiguous genes is observed, corresponding to the deletion of a substantial subsequence from the DNA sequence of a chromosome. Much smaller subsequence deletions may also be observed, leading to abnormal and often nonfunctional genes. A gene deletion may be observed in only one of the two chromosomes of a chromosome pair, in which case a gene deletion is referred to as being hemizygous.

A second chromosomal abnormality in the altered genome shown in FIG. 5 is duplication of genes 430, 431, and 432 in the maternal chromosome C2ₘ′ 507 of the second chromosome pair 506. Duplication of one or more contiguous genes within a chromosome is referred to as gene amplification. In the example altered genome shown in FIG. 5, the gene amplification in chromosome C2ₘ′ is heterozygous, since gene amplification does not occur in the other chromosome of the pair C2ₚ′ 508. The gene amplification illustrated in FIG. 5 is a two-fold amplification, but three-fold and higher-fold amplifications are also observed. An extreme chromosomal abnormality is illustrated with respect to the third chromosome pair (410 in FIG. 4). In the altered genome illustrated in FIG. 5, the entire maternal chromosome 511 has been duplicated from a third chromosome 513, creating a chromosome triplet 510 rather than a chromosome pair. This three-chromosome phenomenon is referred to as a trisomy. The trisomy shown in FIG. 5 is an example of heterozygous gene amplification, but it is also observed that both chromosomes of a chromosome pair may be duplicated, higher-order amplification of chromosomes may be observed, and heterozygous and hemizygous deletions of entire chromosomes may also occur, although organisms with such genetic deletions are generally not viable.

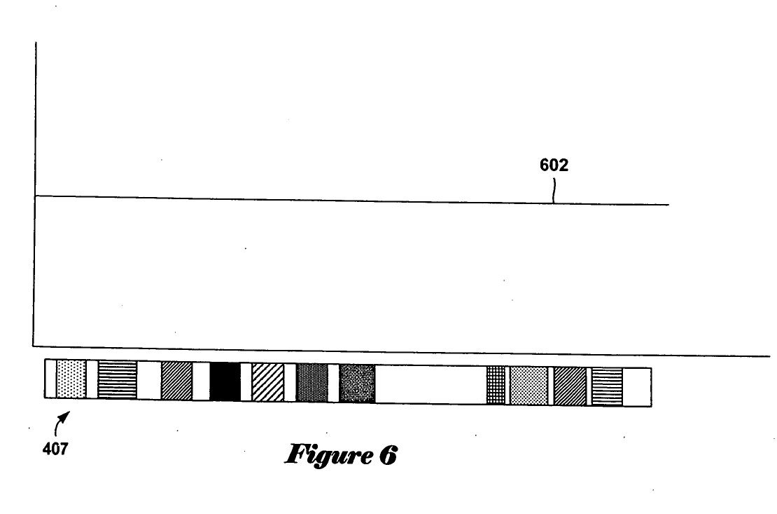

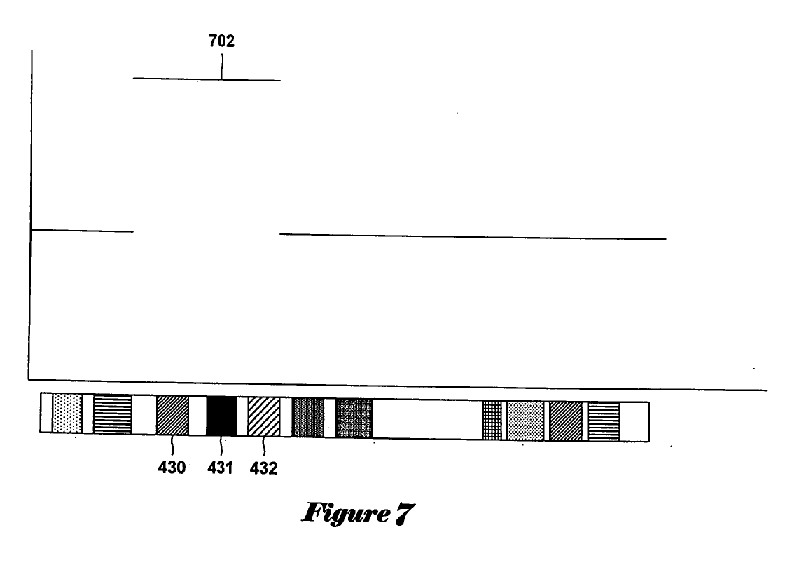

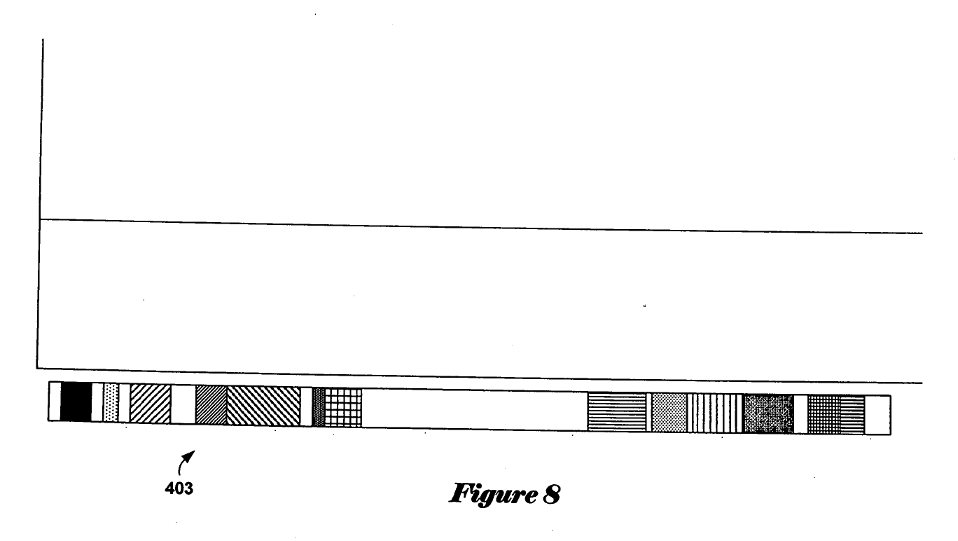

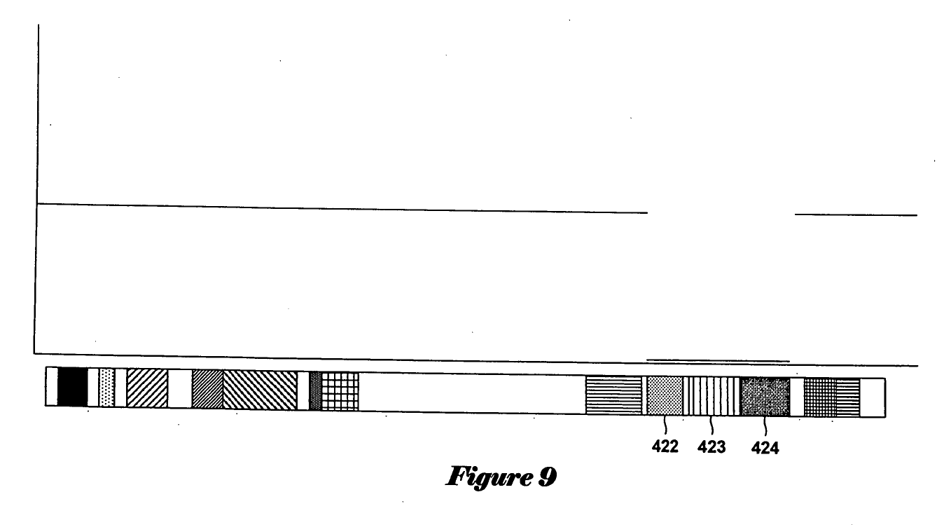

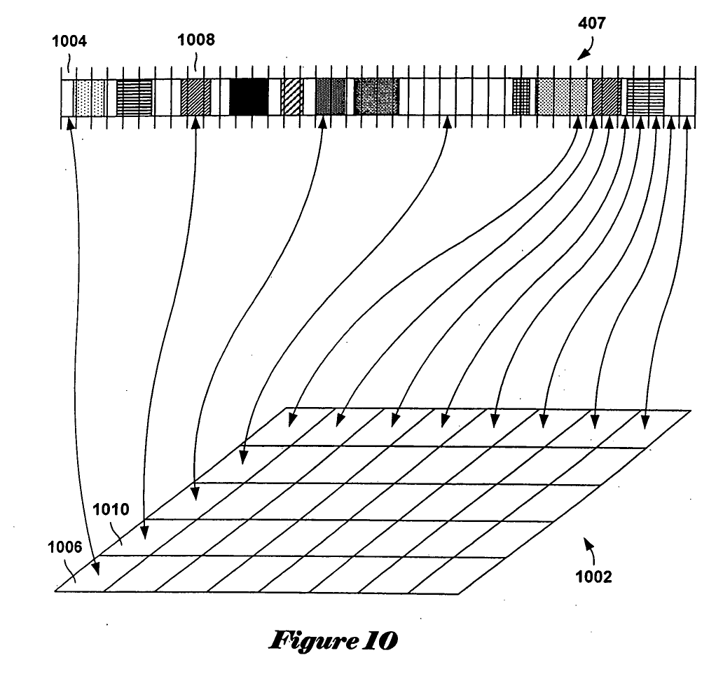

Changes in the number of gene copies, either by amplification or deletion, can be detected by comparative genomic hybridization (“CGH”) techniques. FIGS. 6-7 illustrate detection of gene amplification by CGH, and FIGS. 8-9 illustrate detection of gene deletion by CGH. CGH involves analysis of the relative level of binding of chromosome fragments from sample tissues to single-stranded, normal chromosomal DNA. The tissues-sample fragments hybridize to complementary regions of the normal, single-stranded DNA by complementary binding to produce short regions of double-stranded DNA. Hybridization occurs when a DNA fragment is exactly complementary, or nearly complementary, to a subsequence within the single-stranded chromosomal DNA. In FIG. 6, and in subsequent figures, one of the hypothetical chromosomes of the hypothetical wild-type genome shown in FIG. 4 is shown below the x axis of a graph, and the level of sample fragment binding to each portion of the chromosome is shown along the y axis. In FIG. 6, the graph of fragment binding is a horizontal line 602, indicative of generally uniform fragment binding along the length of the chromosome 407. In an actual experiment, uniform and complete overlap of DNA fragments prepared from tissue samples may not be possible, leading to discontinuities and non-uniformities in detected levels of fragment binding along the length of a chromosome. However, in general, fragments of a normal chromosome isolated from normal tissue samples should, at least, provide a binding-level trend approaching a horizontal line, such as line 602 in FIG. 6. By contrast, CGH data for fragments prepared from the sample genome illustrated in FIG. 5 should generally show an increased binding level for those genes amplified in the abnormal genotype.

FIG. 7 shows hypothetical CGH data for fragments prepared from tissues with the abnormal genotype illustrated in FIG. 5. As shown in FIG. 7, an increased binding level 702 is observed for the three genes 430-432 that are amplified in the altered genome. In other words, the fragments prepared from the altered genome should be enriched in those gene fragments from genes which are amplified. Moreover, in quantitative CGH, the relative increase in binding should be reflective of the increase in a number of copies of particular genes.

FIG. 8 shows hypothetical CGH data for fragments prepared from normal tissue with respect to the first hypothetical chromosome 403. Again, the CGH-data trend expected for fragments prepared from normal tissue is a horizontal line indicating uniform fragment binding along the length of the chromosome. By contrast, the homozygous gene deletion in chromosomes 503 and 504 in the altered genome illustrated in FIG. 5 should be reflected in a relative decrease in binding with respect to the deleted genes. FIG. 9 illustrates hypothetical CGH data for DNA fragments prepared from the hypothetical altered genome illustrated in FIG. 5 with respect to a normal chromosome from the first pair of chromosomes (402 in FIG. 4). As seen in FIG. 9, no fragment binding is observed for the three deleted genes 422, 423, and 424.

CGH data may be obtained by a variety of different experimental techniques. In one technique, DNA fragments are prepared from tissue samples and labeled with a particular chromophore. The labeled DNA fragments are then hybridized with single-stranded chromosomal DNA from a normal cell, and the single-stranded chromosomal DNA then visually inspected via microscopy to determine the intensity of light emitted from labels associated with hybridized fragments along the length of the chromosome. Areas with relatively increased intensity reflect regions of the chromophore amplified in the corresponding tissue chromosome, and regions of decreased emitted signal indicate deleted regions in the corresponding tissue chromosome. In other techniques, normal DNA fragments labeled with a first chromophore are competitively hybridized to a normal single-stranded chromosome with fragments isolated from abnormal tissue, labeled with a second chromophore. Relative binding of normal and abnormal fragments can be detected by ratios of emitted light at the two different intensities corresponding to the two different chromophore labels.

A third type of CGH is referred to as microarray-based CGH (“aCGH”). FIGS. 10-11 illustrate microarray-based CGH. In FIG. 10, synthetic probe oligonucleotides having sequences equal to contiguous subsequences of hypothetical chromosome 407 and/or 408 in the hypothetical, normal genome illustrated in FIG. 4 are prepared as features on the surface of the microarray 1002. For example, a synthetic probe oligonucleotide having the sequence of one strand of the region 1004 of chromosome 407 and/or 408 is synthesized in feature 1006 of the hypothetical microarray 1002. Similarly, an oligonucleotide probe corresponding to subsequence 1008 of chromosome 407 and 408 is synthesized to produce the oligonucleotide probe molecules of feature 1010 of microarray 1002. In actual cases, probe molecules may be much shorter relative to the length of the chromosome, and multiple, different, overlapping and non-overlapping probes/features may target a particular gene. Nonetheless, there is generally a definite, well-known correspondence between microarray features and genes, with the term “genes,” as discussed above, referring broadly to any biopolymer subsequence of interest. There are many different types of aCGH procedures, including the two-chromophore procedure described above, single-chromophore CGH on single-nucleotide-polymorphism arrays, bacterial-artificial-chromosome-based arrays, and many other types of aCGH procedures. The present invention is applicable to all aCGH variants. For each variant, data obtained by comparing signals generated by the variant with signals generated by a normal reference generally constitute a starting point for aCGH analysis. When single-dye technologies are used, multiple microarray-based procedures may be needed for aCGH analysis.

The microarray may be exposed to sample solutions containing fragments of DNA. In one version of aCGH, an array may be exposed to fragments, labeled with a first chromophore, prepared from potentially abnormal tissue as well as to fragments, labeled with a second chromophore, prepared from a normal or control tissue. The normalized ratio of signal emitted from the first chromophore versus signal emitted from the second chromophore for each feature provides a measure of the relative abundance of the portion of the normal chromosome corresponding to the feature in the abnormal tissue versus the normal tissue. In the hypothetical microarray 1002 of FIG. 10, each feature corresponds to a different interval along the length of chromosome 407 and 408 in the hypothetical wild-type genome illustrated in FIG. 4. When fragments prepared from a normal tissue sample, labeled with a first chromophore, and DNA fragments prepared from normal tissue labeled with the second chromophore, are both hybridized to the hypothetical microarray shown in FIG. 10, and normalized intensity ratios for light emitted by the first and second chromophores are determined, the normalized ratios for all features should be relatively uniformly equal to one.

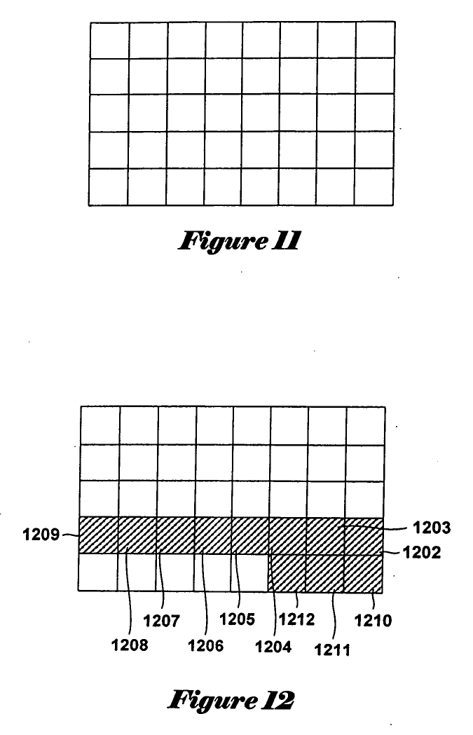

FIG. 11 represents an aCGH data set for two normal, differentially labeled samples hybridized to the hypothetical microarray shown in FIG. 10. The normalized ratios of signal intensities from the first and second chromophores are all approximately unity, shown in FIG. 11, by log ratios for all features of the hypothetical microarray 1002 displayed in the same color. By contrast, when DNA fragments isolated from tissues having the abnormal genotype, illustrated in FIG. 5, labeled with a first chromophore are hybridized to the microarray, and DNA fragments prepared from normal tissue, labeled with a second chromophore, are hybridized to the microarray, then the ratios of signal intensities of the first chromophore versus the second chromophore vary significantly from unity in those features containing probe molecules equal to, or complementary to, subsequences of the amplified genes 430, 431, and 432. As shown in FIG. 12, increase in the ratio of signal intensities from the first and second chromophores, indicated by darkened features, are observed in those features 1202-1212 with probe molecules equal to, or complementary to, subsequences spanning the amplified genes 430, 431, and 432. Similarly, a decrease in signal intensity ratios indicates gene deletion in the abnormal tissues.

Microarray-based CGH data obtained from well-designed microarray experiments provide a relatively precise measure of the relative or absolute number of copies of genes in cells of a sample tissue. Sets of aCGH data obtained from pre-cancerous and cancerous tissues at different points in time can be used to monitor genome instability in particular pre-cancerous and cancerous tissues. Quantified genome instability can then be used to detect and follow the course of particular types of cancers. Moreover, quantified genome instabilities in different types of cancerous tissue can be compared in order to elucidate common chromosomal abnormalities, including gene amplifications and gene deletions, characteristic of different classes of cancers and pre-cancerous conditions, and to design and monitor the effectiveness of drug, radiation, and other therapies used to treat cancerous or pre-cancerous conditions in patients. Unfortunately, biological data can be extremely noisy, with the noise obscuring underlying trends and patterns. Scientists, diagnosticians, and other professionals have therefore recognized a need for statistical methods for normalizing and analyzing aCGH data, in particular, and CGH data in general, in order to identify signals and patterns indicative of chromosomal abnormalities that may be obscured by noise arising from many different kinds of experimental and instrumental variations.

One approach to ameliorating the effects of high noise levels in CGH data involves normalizing sample-signal data by using control signal data. Features can be included in a microarray to respond to genome targets known to be present at well-defined multiplicities in both sample genome and the control genome. Control signal data can be used to estimate an average ratio for abnormal-genome-signal intensities to control-genome-signal intensities, and each abnormal-genome signal can be multiplied by the inverse of the estimated ratio, or normalization constant, to normalize each abnormal-genome signal to the control-genome signals. Another approach is to compute the average signal intensity for the abnormal-genome sample and the average signal intensity for the control-genome sample, and to compute a ratio of averages for abnormal-genome-signal intensities to control-genome-signal intensities based on averaged signal intensities for both samples.

In a more general case, an aCGH array may contain a number of different features, each feature generally containing a particular type of probe, each probe targeting a particular chromosomal DNA subsequence indexed by index k that represents a genomic location. A subsequence indexed by index k is referred to as “subsequence k.” One can define the signal generated for subsequence k as the sum of the normalized log-ratio signals from the different probes targeting subsequence k divided by the number of probes targeting subsequence k or, in other words, the average log-ratio signal value generated from the probes targeting subsequence k, as follows:

$$C ( k ) = \frac{{\sum }_{b ∈ \{ features containing probes for k \}} C ( b )}{{num\_features}_{k}}$$

where num_featuresₖ is the number of features that target the subsequence k; and C(b) is the normalized log-ratio signal measured for feature b,

$$C ( b ) = {log ( \frac{{I}_{red}}{{I}_{green}} )}_{b} - \frac{{\sum }_{i ∈ \{ all features \}} {log ( \frac{{I}_{red}}{{I}_{green}} )}_{i}}{num\_features}$$

In the case where a single probe targets a particular subsequence, k, no averaging is needed. In the following discussion, normalization of signals for a solution of interest is discussed, such as a solution of DNA fragments obtained from a particular tissue or experiment. A solution of interest may be subject to a single CGH analysis, or a number of identical samples derived from the solution of interest may be each separately subject to CGH analysis, and the signals produced by the analysis for each subsequence k may be averaged to produce a single, averaged, signal data set for the solution of interest.

To re-emphasize, each aCGH data point is generally a log ratio of signals read from a particular feature of a microarray that contains probes targeting a particular subsequence, the log-ratio of signals representing the ratio of signals emitted from a first label used to label fragments of a genome sample to a signal generated from a second label used to label fragments of a normal, control genome. Both the sample-genome fragments and the normal, control fragments hybridize to normal-tissue-derived probe molecules on the microarray. A normal tissue or sample may be any tissue or sample selected as a control tissue or sample for a particular experiment. The term “normal” does not necessarily imply that the tissue or sample represents a population average, a non-diseased tissue, or any other subjective or object classification. The sample genome may be obtained from a diseased or cancerous tissue, in order to compare the genetic state of the diseased or cancerous tissue to a normal tissue, but may also be a normal tissue.

Subsequence deletions and amplifications generally span a number of contiguous subsequences of interest, such as genes, control regions, or other identified subsequences, along a chromosome. It therefore makes sense to analyze aCGH data in a chromosome-by-chromosome fashion, statistically considering groups of consecutive subsequences along the length of the chromosome in order to more reliably detect amplification and deletion. Specifically, it is assumed that the noise of measurement is independent for each subsequence along the chromosome, and independent for distinct probes. Statistical measures are employed to identify sets of consecutive subsequences for which deletion or amplification is relatively strongly indicated. This tends to ameliorate the effects of spurious, single-probe anomalies in the data. This is an example of an aberration-calling technique, in which gene-copy anomalies appearing to be above the data-noise level are identified.

One can consider the measured, normalized, or otherwise processed signals for subsequences along the chromosome of interest to be a vector V as follows:

V={ν₁, ν₂, . . . , νₙ}

where νₖ=C(k)
Note that the vector, or set V, is sequentially ordered by position of subsequences along the chromosome. A statistic S is computed for each interval I of subsequences along the chromosome as follows:

$$S ( I ) = ( {\sum }_{k = i , … , j} {v}_{k} ) \cdot \frac{1}{\sqrt{j - i + 1}}$$

where

$$I = \{ {v}_{i} , … , {v}_{j} \} ; and$$

$${v}_{k} = C ( k )$$

Under a null model assuming no sequence aberrations, the statistic S has a normal distribution of values with mean=0 and variance=1, independent of the number of probes included in the interval I. The statistical significance of the normalized signals for the subsequences in an interval I can be computed by a standard probability calculation based on the area under the normal distribution curve:

$$Prob ( \vert S ( I ) \vert > z ) \approx ( \frac{1}{\sqrt{2 π}} ) \frac{1}{z} {e}^{- \frac{{z}^{2}}{2}}$$

Alternatively, the magnitude of S(I) can be used as a basis for determining alteration.

It should be noted that various different interval lengths may be used, iteratively, to compute amplification and deletion probabilities over a particular biopolymer sequence. In other words, a range of interval sizes can be used to refine amplification and deletion indications over the biopolymer.

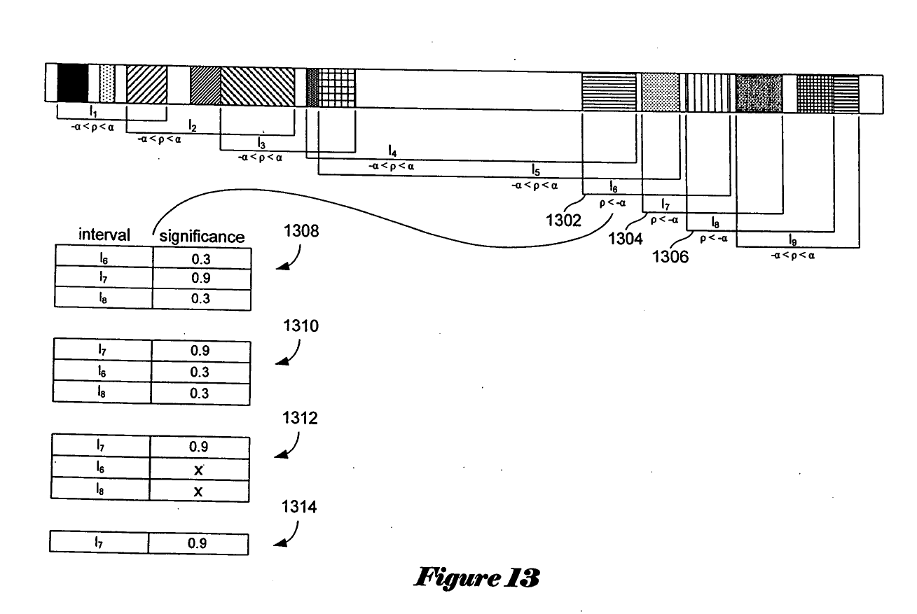

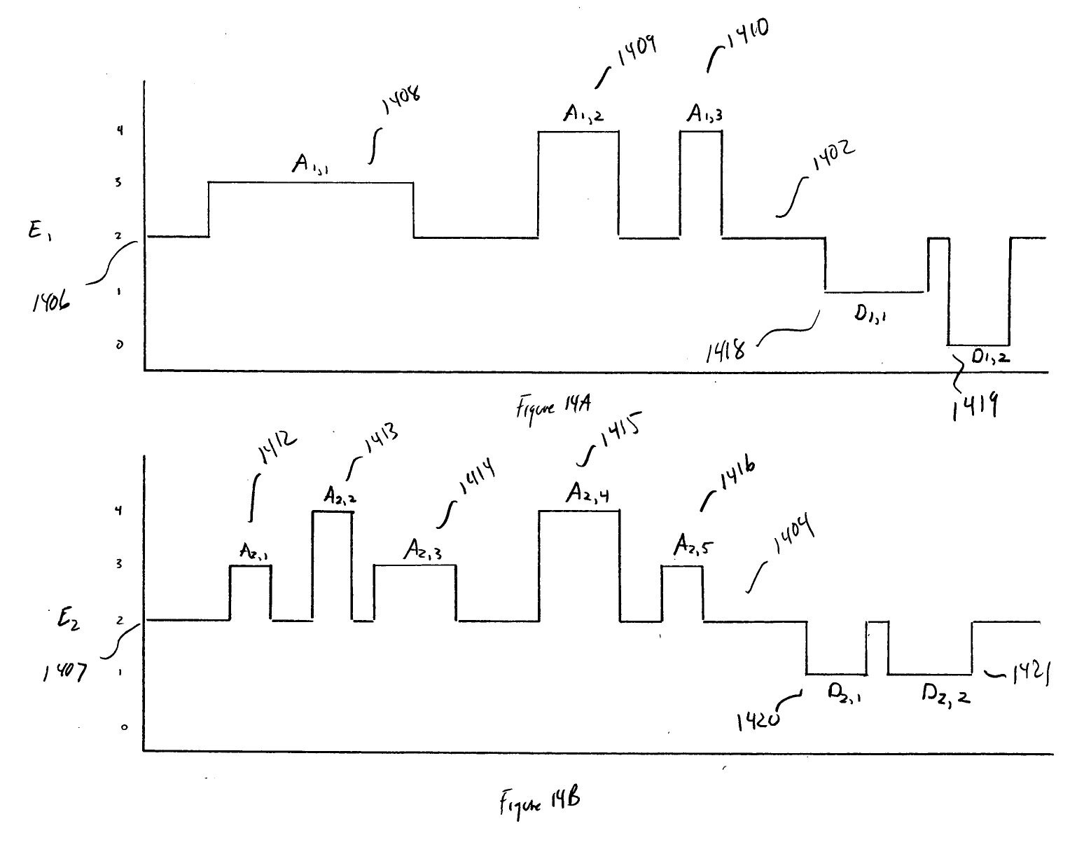

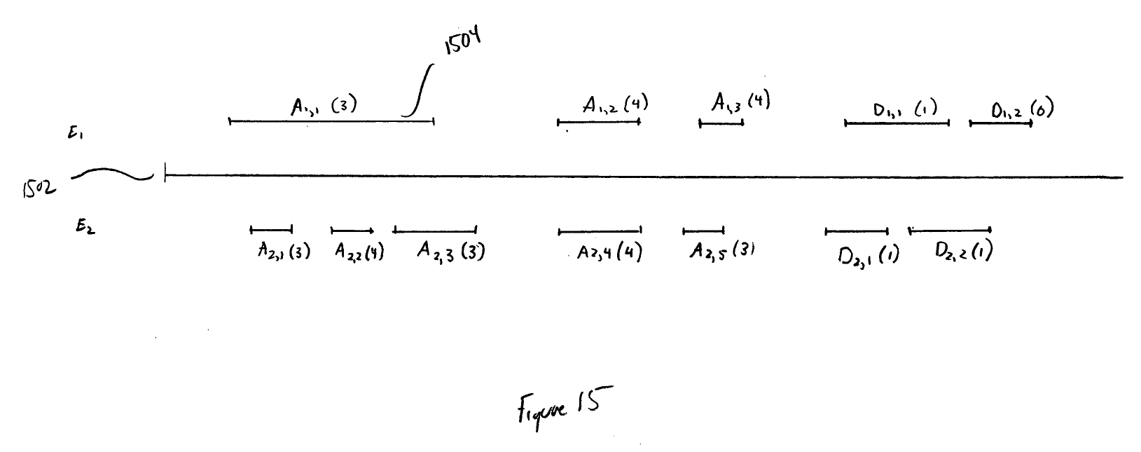

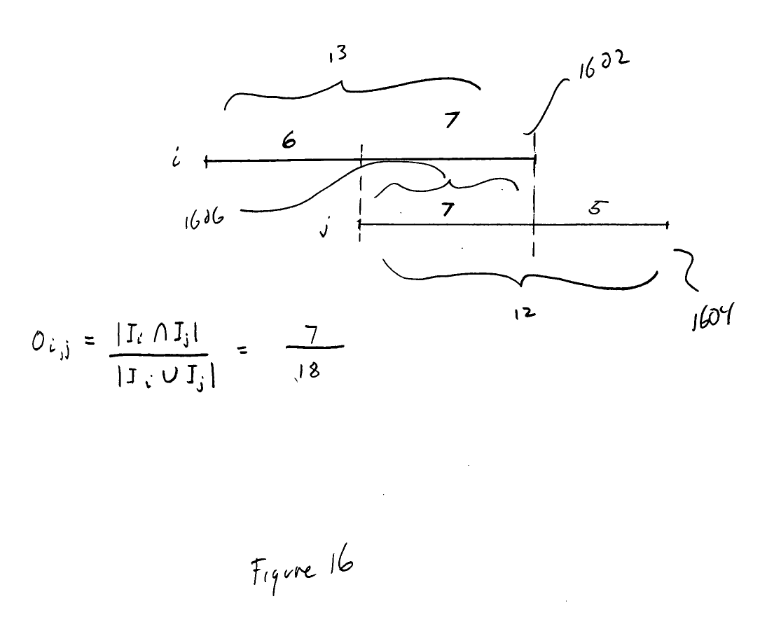

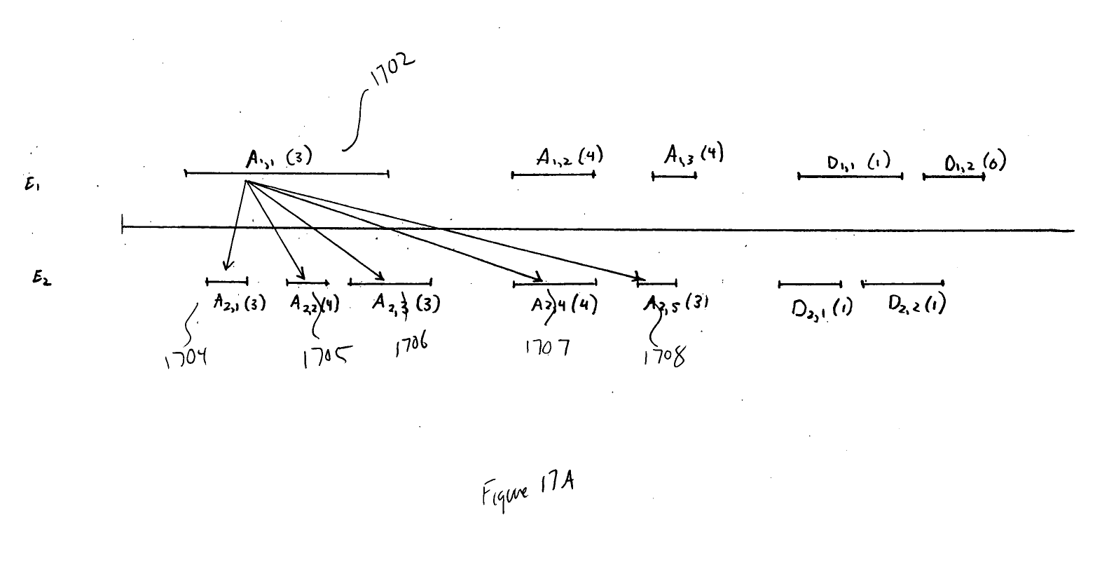

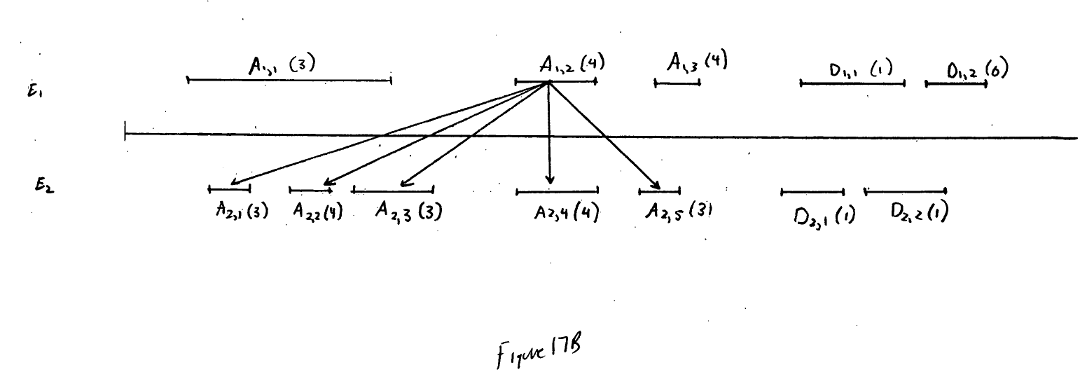

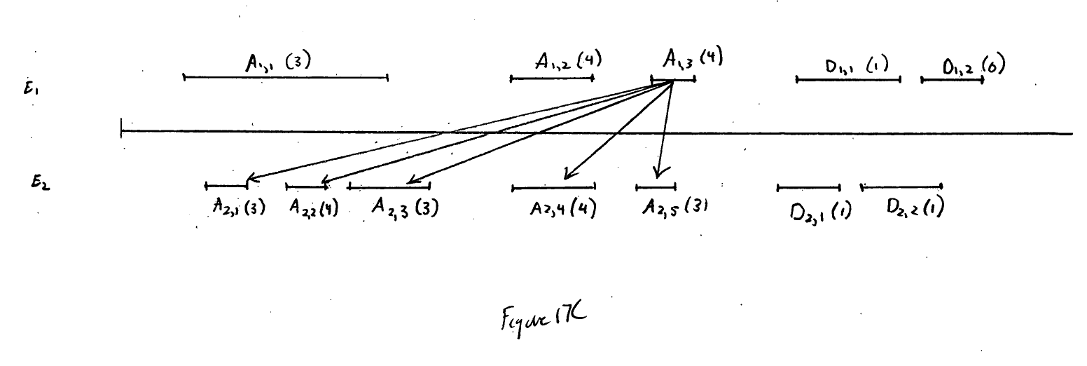

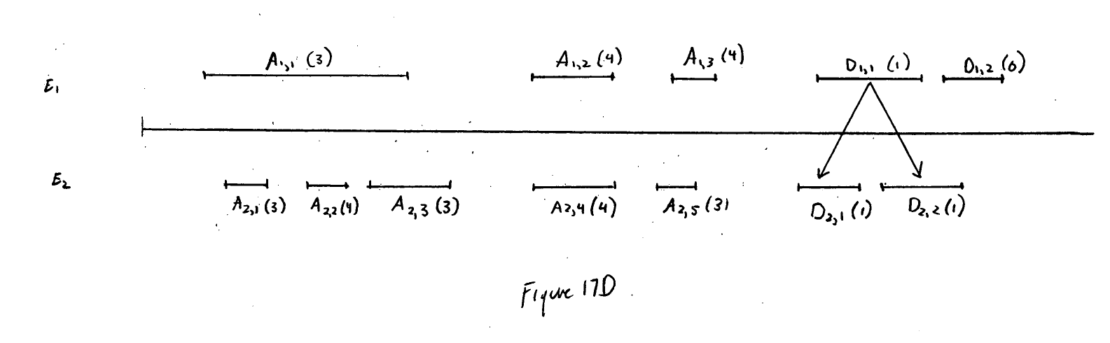

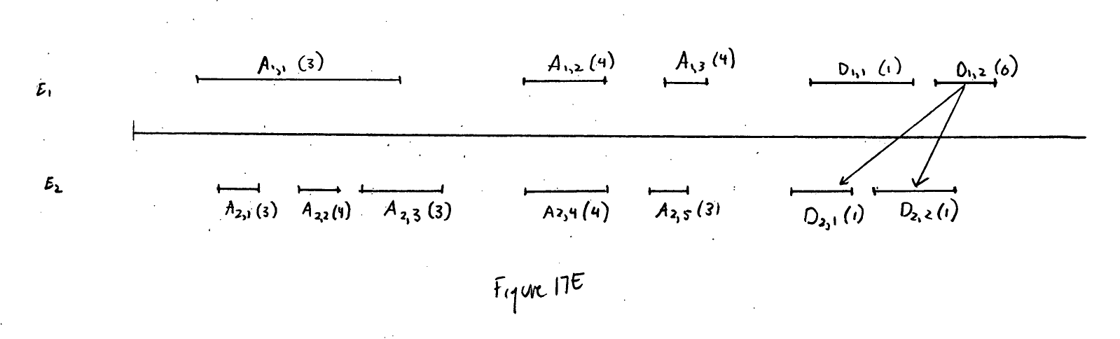

After the probabilities for the observed values for intervals are computed, those intervals with computed probabilities outside of a reasonable range of expected probabilities under the null hypothesis of no amplification or deletion are identified, and redundancies in the list of identified intervals are removed. FIG. 13 illustrates one method for identifying and ranking intervals and removing redundancies from lists of intervals identified as corresponding to probable deletions or amplifications. In FIG. 13, the intervals for which probabilities are computed along the chromosome C₁ (402 in FIG. 4) for diseased tissue with an abnormal chromosome (502 in FIG. 5) are shown. Each interval is labeled by an interval number, Iₓ, where x ranges from 1 to 9. For most intervals, the calculated probability falls within a range of probabilities consonant with the null hypothesis. In other words, neither amplification nor deletion is indicated for most of the intervals. However, for intervals I₆ 1302, I₇, 1304, and I₈, 1306, the computed probabilities fall below the range of probabilities expected for the null hypothesis, indicating potential subsequence deletion in the diseased-tissue sample. These three intervals are placed into an initial list 1308 which is ordered by the significance of the computed probability into an ordered list 1310. Note that interval I₇ 1304 exactly includes those subsequences deleted in the diseased-tissue chromosome (502 in FIG. 5), and therefore reasonably has the highest significance with respect to falling outside the probability range of the null hypothesis. Next, all intervals overlapping an interval occurring higher in the ordered list are removed, as shown in list 1312, where overlapping intervals I₆ and I₈, with less significance, are removed, as indicated by the character X placed into the significance column for the entries corresponding to intervals I₆ and I₈. The end result is a list containing a single interval 1314 that indicates the interval most likely coinciding with the deletion. The final list for real chromosomes, containing thousands of subsequences and analyzed using hundreds of intervals, may generally contain more than a single entry. Additional details regarding computation of interval scores can be found in “Efficient Calculation of Interval Scores for DNA Copy Number Data Analysis,” Lipson et al., Proceedings of RECOMB 2005, LNCS 3500, p. 83, Springer-Verlag.

### EMBODIMENTS OF THE PRESENT INVENTION

Method and system embodiments of the present invention may be used to evaluate the quality of data obtained in aCGH experiments. In certain embodiments of the present invention, interval-based aberration-calling methods outlined in the previous subsection are employed to determine regions of amplification and deletion in a chromosome or chromosomal region analyzed by aCGH experiments. The products of the aberration-calling methods are indications of the relative abundance of subsequences of a sample genome with respect to a control genome after the signal data has been normalized and analyzed by an aberration-calling method that identifies indications of subsequence deletion and amplifications that are significant with respect to signal noise. The quality of an experimental result may refer to the reproducibility of the result, accuracy of the result, precision of the result, and other such characteristics. In the following discussion, the measured quality is the similarity between sets of aberrations called out by aberration-detecting analysis of either identically-executed or similar aCGH experiments, each set of aberrations derived from a separate aCGH experiment or experiments. Similarity between sets of aCGH experimental results may be directly or indirectly reflective of reproducibility, accuracy, and precision, and may also be indirectly reflective of the reproducibility, accuracy, and precision of underlying sample preparation and biological and biochemical protocols, array-based experimental technique, collection of data from microarrays, and analysis of microarray-derived data.

Currently, many different measures of intra-array quality and consistency are used to ascertain the quality of aCGH experimental results. These intra-array quality and consistency measurements include measurements of signal-to-noise ratios of selected or averaged signals read from array elements, average background signals, statistical measures of signals produced by negative control probes, and other such quality and consistency measures based on signals measured for sets of array elements. These intra-array quality measurements are, in other words, based on relatively low-level data far below eventual biologically related and genomically related results derived from signals and signal statistics measured from array elements. Moreover, it may be difficult to employ intra-array quality measurements in order to measure or determine the overall quality of a series of array-based experiments. Most importantly, when multiple experimental results provide for redundant data, it is desirable to take advantage of the redundant data to measure and improve data quality.

The present invention provides a variety of new, inter-array quality measurements based on comparison of high-level analytical results derived from multiple array-based experiments. The present invention can also be employed to measure the quality of multiple CGH experimental results obtained from other types of CGH analysis. In general, identically-executed experiments, referred to as “replicate experiments,” or very similar experiments, such as dye-flip experiments in which the sense of chromophore labels is reversed between different experiments in two-channel experiments, or multiple different chromophore-to-experimental-component assignments are used in multiple multi-channel experiments, may be evaluated by method embodiments of the present invention. The quality metrics determined by embodiments of the present invention are based on high-level analytical results, rather than signals measured from individual array elements or statistics computed from sets of array-element measurements, and are therefore potentially more robust and less sensitive to less relevant variations in low-level measured signals. Moreover, the quality metrics produced by various embodiments of the present invention inherently involve multiple experiments, and are thus useful in evaluating the overall quality of a set of identically-executed or similar experiments.

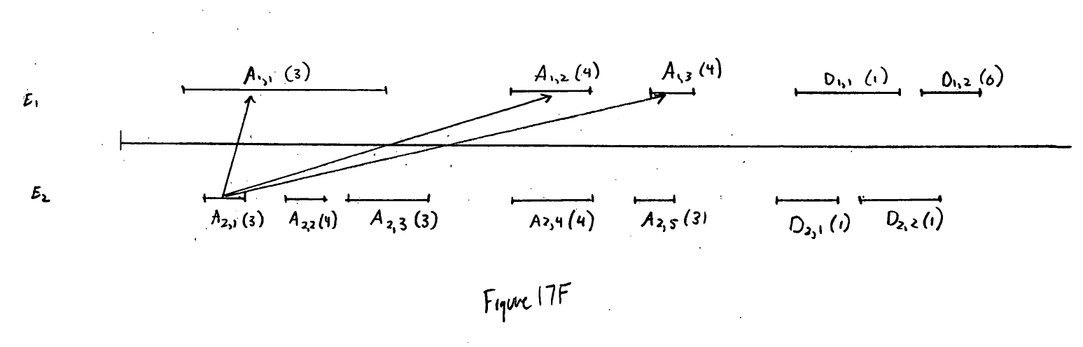

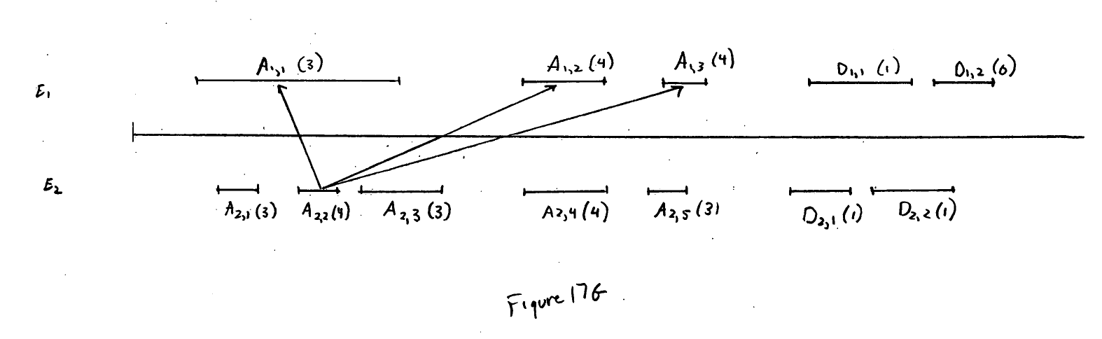

FIGS. 14A-B illustrate two hypothetical aCGH experimental results. FIG. 14A shows a plot 1402 of copy number, with respect to the vertical axis, versus chromosome position, with respect to the horizontal axis, that represents an aCGH experiment E₁ in which amplification and deletion aberrations, also referred to as “amplification and deletion intervals,” are determined for a sample chromosome with respect to a control, or normal, chromosome. FIG. 14B shows a plot 1404 of copy number versus chromosomal position for an identically-executed aCGH experiment E₂. The aCGH experiment E₁ may have been carried out on a first microarray, and the aCGH experiment E₂ may have been identically carried out on a second microarray using different portions of the sample, or identically prepared samples. Alternatively, aCGH experiments E₁ and E₂ may have been carried out similarly, with the sense of chromophore labels used in the experiments flipped, or interchanged, in the two experiments. For example, the red label may be used for the sample chromosome, and the green label may be used for the control in experiments E₁ while the green label may be used for the sample chromosome, and the red label may be used for the control in experiments E₂. The horizontal line corresponding to a measured copy number of 2 (1406 and 1407 in plots 1402 and 1404, respectively) represents a normal copy number for both of the hypothetical experiments E₁ and E₂. Amplification aberrations are regions of the chromosome with copy number greater than 2, including amplification aberrations 1408-1410 in plot 1402 and amplification aberrations 1412-1416 in plot 1404. Deletion aberrations are regions of the chromosome with measured copy number less than 2, including deletion aberrations 1418 and 1419 in plot 1402 and deletion aberrations 1420 and 1421 in plot 1404. Note that amplifications and deletions are referred to using the notation A_{x,n} and B_{x,n} where the subscript x refers to the numerical index of the experiment or experimental result and the subscript n refers to the sequential number of the amplification or deletion along the chromosome. Note also that, in general, an actual aCGH experiment might produce many hundreds or thousands of amplifications and deletions. The hypothetical experimental results shown in FIGS. 4A-B are vastly simplified plots used for illustration purposes only.

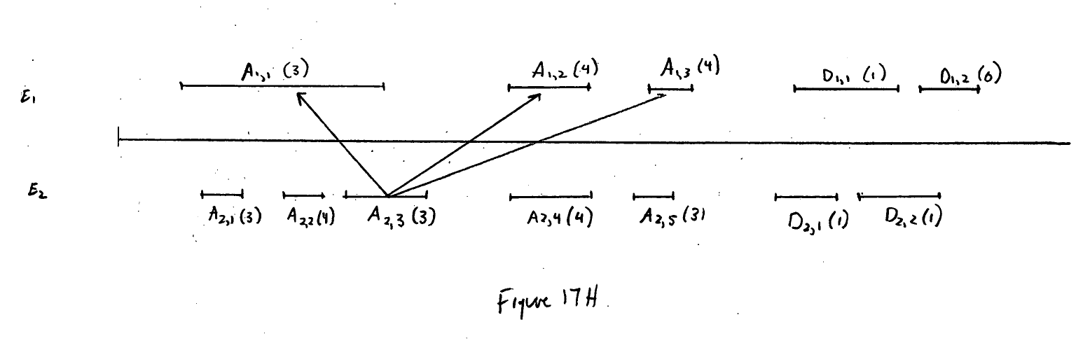

FIG. 15 shows an alternative graphical representation of the two experimental results E₁ and E₂. In FIG. 15, the amplifications and deletions observed in experimental results E₁ and E₂ are shown positioned above and below, respectively, a horizontal line 1502 representing the chromosome analyzed by aCGH experiments E₁ and E₂. Each interval representing an amplification or deletion, such as interval 1504, is annotated with the amplification or deletion label as well as with a copy-number value in parenthesis. Thus, interval 1504 in FIG. 15 corresponds to amplification A_{1,1} 1408 in FIG. 14A.

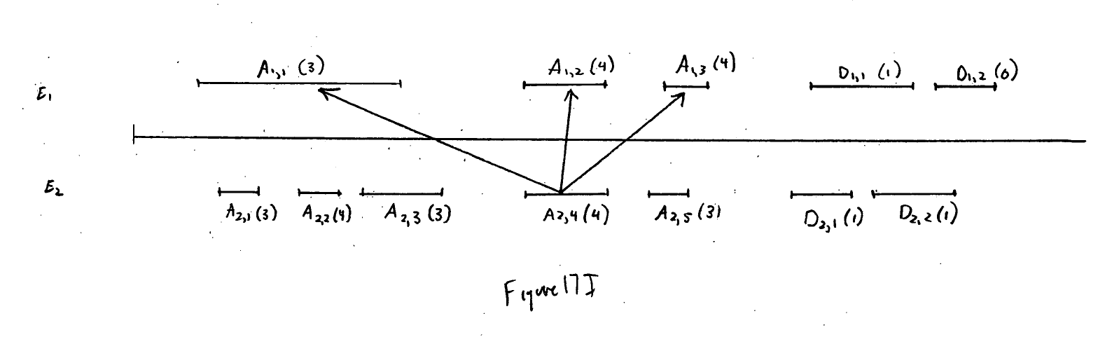

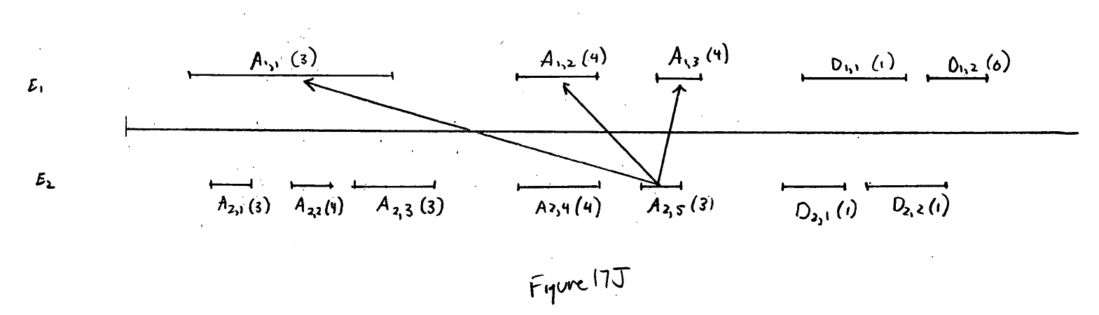

In a first method embodiment of the present invention, an overlap is computed, bi-directionally, for each amplification and deletion in the first experimental result E₁ with respect to the second experimental result E₂, and for each amplification and deletion in the second experimental result E₂ with respect to the first experimental result E₁. FIG. 16 illustrates calculation, according to a method embodiment of the present invention, of an interval-overlap metric O_{i,j} based on two aberrant intervals i and j representing either two amplifications or two deletions within the two different experiments results E₁ and E₂. FIG. 16 uses illustration conventions similar to those of FIG. 15. The interval i 1602 has a length, in probes or in an arbitrary number-of-base-pairs units, of 13 and the interval j 1604 has a length, in probes or number-of-base-pairs units, of 12. The interval i positionally overlaps the interval j for a length of 7 1606. The overlap metric O_{i,j} may be computed as:

$${O}_{i , j} = \frac{\vert {I}_{i} ⋂ {I}_{j} \vert}{\vert {I}_{i} ⋃ {I}_{j} \vert} = \frac{7}{18}$$

where |Iᵢ∩Iⱼ| is the length in probes, or number-of-base-pairs units, of the intersection, or overlap region, between intervals i and j and |Iᵢ∪Iⱼ| is the total combined lengths of intervals i and j. The overlap metric O_{i,j} ranges from 0, when intervals i and j do not overlap positionally with respect to the measured chromosome, and 1, when intervals i and j are of the same length and are identically positioned with respect to the chromosome.

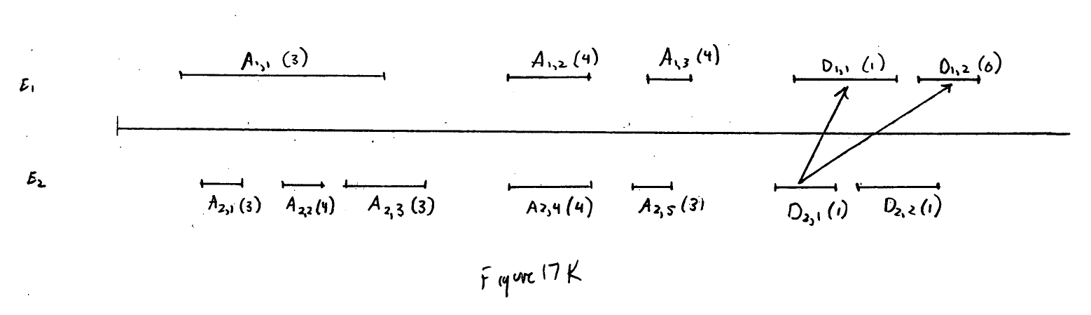

Two experimental results E₁ and E₂ can be compared by producing a pairwise overlap metric O(E₁,E₂) for the two experimental results. FIGS. 17A-L illustrate computation of a pairwise overlap metric O(E₁, E₂) for the experimental results E₁ and E₂ shown in FIGS. 14A-B, according to a first described method embodiment of the present invention. As shown in FIG. 17A, the first amplification interval A_{1,1} 1702 of experimental result E₁ is compared to each of the amplification intervals 1704-1708 of the experimental result E₂ by computing interval-overlap metrics O_{A}_{_{1,1}}_{,A}_{_{2,1}}, where i ranges from 1 to 5. The maximum of these computed overlap metrics O_{A}_{_{1,1}}_{,A}_{_{2,j}} is selected as the computed overlap for amplification A_{1,1}. Similarly, as shown in FIGS. 17B-C, the maximum overlaps computed for amplification intervals A_{1,2} and A_{1,3} with respect to the amplification intervals and experimental results E₂ are determined and added to the maximum overlap metric computed for interval A_{1,1} in FIG. 17A. This sum constitutes the first of four terms computed for the overall overlap O(E₁,E₂) computation. Next, as shown in FIG. 17D-E, maximum overlap intervals are computed and summed together for E₁ deletion intervals D_{1,1} and D_{1,2}. This sum constitutes the second of four terms computed in order to compute the overall overlap O(E₁,E₂). Then, as shown in FIGS. 17F-J, maximum overlap metrics O_{A}_{_{2,j}}_{,A}_{_{1,j}},where i ranges from 1 to 3 and j ranges from 1 to 5, are computed for each of the amplification intervals in experimental result E₂ with respect to experimental result E₁ and summed together to produce the third of four terms computed in order to compute the overall overlap O(E₁,E₂). Finally, as shown in FIGS. 17K-L, maximum overlap metrics are computed for each of the deletions D_{2,1} and D_{2,2} in experimental result E₂ with respect to experimental result E₁ to produce the fourth of four terms computed in order to compute the overall overlap O(E₁,E₂). The four terms are summed, and then divided by the length of aberrant intervals in experimental results E₁ and E₂, |E₁|+|E₂|, where length can be computed in numbers of base pairs in the aberrant intervals, number of probes directed to the aberrant intervals, or other measures of interval length, to produce the final overall overlap metric O(E₁,E₂). In mathematical notation: O_{i,j}=interval-overlap which ranges from 1 to 0

$${E}_{1} = \{ {A}_{1 , 1} , {A}_{1 , 2} , … , {A}_{1 , m} , {D}_{1 , 1} , {D}_{1 , 2} … , {D}_{1 , n} \}$$

$${E}_{2} = \{ {A}_{2 , 1} , {A}_{2 , 2} , … {A}_{2 , p} , {D}_{2 , 1} , {D}_{2 , 2} , … , {D}_{2 , q} \}$$

$$O ( {E}_{1} , {E}_{2} ) = \frac{[ \begin{matrix}{\sum }_{i = 1}^{m} {max}_{j = 1 to p} ( {O}_{{A}_{1 , j} , {A}_{2 , j}} ) + {\sum }_{i = 1}^{n} {max}_{j = 1 to q} ( {O}_{{D}_{1 , j} , {D}_{2 , j}} ) + \\ {\sum }_{i = 1}^{p} {max}_{j = 1 to m} ( {O}_{{A}_{2 , j} , {A}_{1 , j}} ) + {\sum }_{i = 1}^{q} {max}_{j = 1 to n} ( {O}_{{D}_{2 j}} {,}_{{D}_{1 , j}} )\end{matrix} ]}{\vert {E}_{1} \vert + \vert {E}_{2} \vert}$$

$$where 0 \leq O ( {E}_{1} , {E}_{2} ) \leq 1$$

In various implementations of the method embodiments of the present invention, to improve computational efficiency, all interval-overlap metrics may not need to be computed when it can be determined that two intervals do not overlap from their respective starting and ending positions. Instead, for each term, only a subset of the interval-overlap metrics may need to be computed, and a maximum chosen from the subset of the interval-overlap metrics.

In the case that an overall overlap metric needs to be computed for a set of experimental results of cardinality greater than 2, then an overall overlap metric O(ε) can be computed from the set of experimental results ε={E₁, . . . Eₖ} by summing all pairwise overlap metrics and then normalizing the sum, as follows:

$$ɛ = \{ {E}_{1} , … {E}_{k} \}$$

$$O ( ɛ ) = \frac{2}{k ( k - 1 )} {\sum }_{i = 1}^{k} {\sum }_{j = i + 1}^{k} O ( {E}_{i} , {E}_{j} )$$

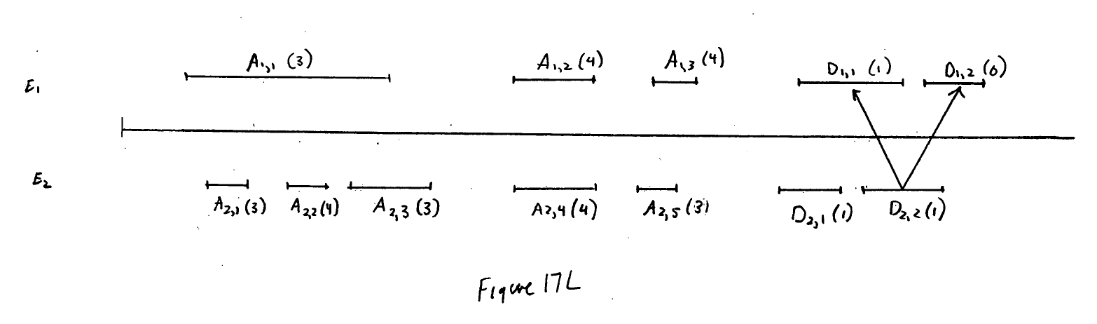

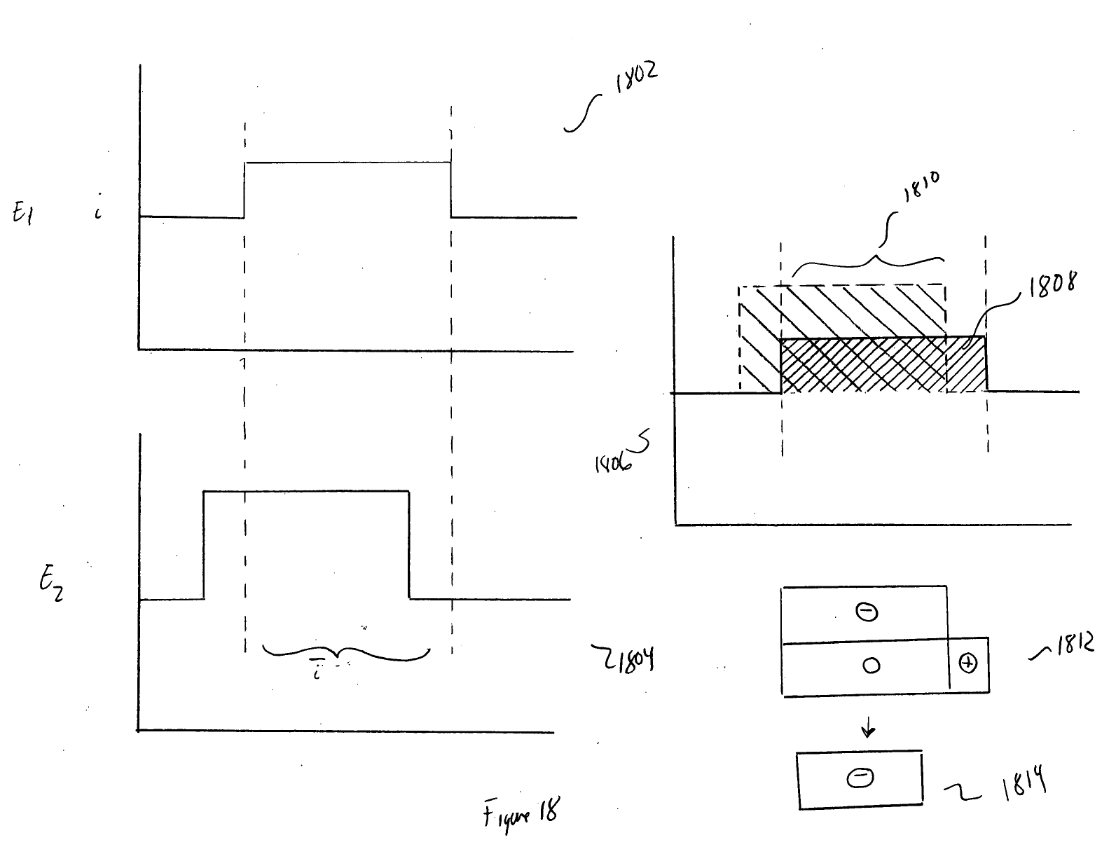

In an alternative method embodiment of the present invention, an alternative overlap metric Oᵢ′ may be computed for each interval in a first experimental result with respect to a second experimental result. FIG. 18 illustrates computation of the alternative interval-overlap metric Oᵢ′ according to a method embodiment of the present invention. In FIG. 18, two small portions of experimental results E₁ and E₂ are plotted as plots 1802 and 1804. The experimental results are both aligned to a common chromosomal position. Computation of the overlap metric Oᵢ′ involves, as shown in combined plot 1806, subtracting from the area of aberrant interval A_{1,i} 1808 the area of the corresponding signal for the same interval i in experimental results E₂ 1810. The subtraction can be diagrammatically represented 1812 to produce a relatively small, negative area 1814. The absolute value of the subtraction can be used in order to produce a range of metric values from 0, indicating complete overlap, to a number that depends on the areas of the signals for interval i in E₁ and E₂ and that increases in value as the signals in the two experimental results diverge from one another. Thus, the alternative overlap metric Oᵢ′ can be expressed as:

O′=|signal_{E1}(i)−signal_{E2}(i)|

where i is a particular interval, signa_{E1}(i) is the area of the signal for interval i in experimental results E₁ and signal_{E2} is the area of the signal for interval i in experimental results E₂. In this case, a pairwise overlap metric O(E₁,E₂) can be computed for two experimental results E₁ and E₂ as follows:

$${E}_{1} = \{ {A}_{1 , 1} , {A}_{1 , 2} , … {A}_{1 , m} , {D}_{1 , 1} , {D}_{1 , 2} , … {D}_{1 , n} \}$$

$${E}_{2} = \{ {A}_{2 , 1} , {A}_{2 , 2} , … , {A}_{2 , p} , {D}_{2 , 1} , {D}_{2 , 2} , … , {D}_{2 , q} \}$$

$$O ( {E}_{1} , {E}_{2} ) = {\sum }_{i = 1}^{m} {O}_{{A}_{1 j}}^{′} + {\sum }_{i = 1}^{n} {O}_{{D}_{1 j}}^{′} + {\sum }_{i = 1}^{p} {O}_{{A}_{2 j}}^{′} + {\sum }_{i = 1}^{q} {O}_{{D}_{2 j}}^{′}$$

The computation of the difference between signals, as shown in FIG. 18, can be carried out in a variety of ways. In still alternative embodiments, other types of simple, arithmetic comparisons between the signal in an interval of one experimental result and the signal in a corresponding interval in a second experimental result can be used to provide a range of values, with identical signals producing one extreme value and completely divergent signals producing values at the opposite end of the range. The alternative overall overlap metric O(E₁,E₂) computed with the alternative overlap metric Oᵢ′ can be used for computing an overall overlap metric O(ε) for sets of more than two experimental results, as discussed above.

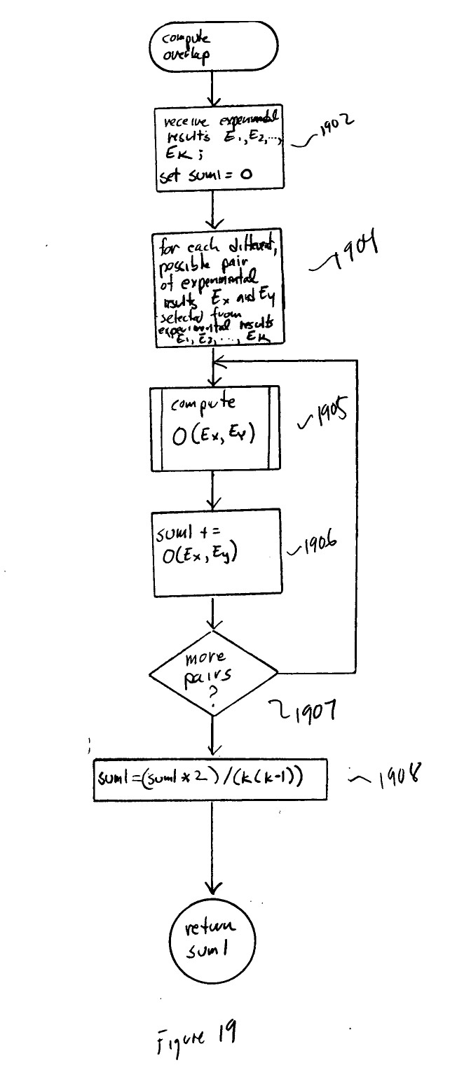

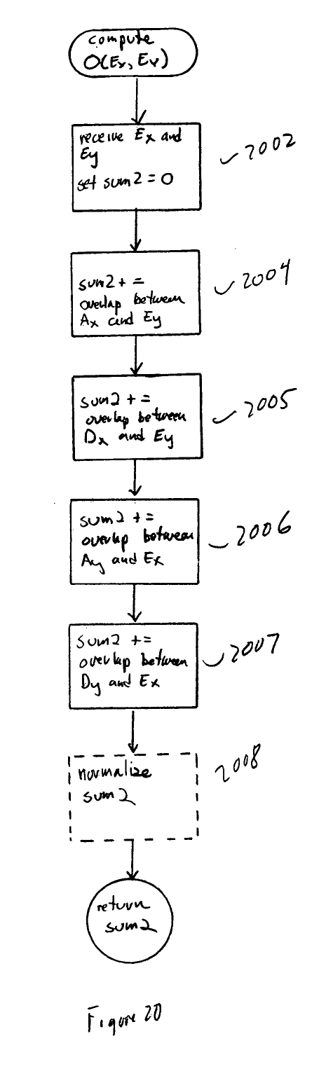

FIGS. 19 and 20 are control-flow diagrams representing a quality-metric calculation for a set of k experimental results according to embodiments of the present invention. FIG. 19 illustrates computation of the overall overlap O(ε), discussed above. In step 1902, the routine “compute overlap” receives the set of experimental results {E₁, E₂, . . . , Eₖ}. In addition, the local variable sum1 is set to 0. In the for-loop of steps 1904-1907, pairwise overlap metrics O(Eₓ,E_{y}) are computed for each possible pair of experimental results Eₓ and E_{y} selected from the k experimental results received in step 1902, and are accumulated in the local variable sum1, in step 1906. Finally, in step 1908, the value in local variable sum1 is optionally normalized, as discussed above. The contents of local variable sum1 are returned as the computed quality metric.

FIG. 20 illustrates computation of the pairwise overlap metric O(Eₓ,E_{y}) in step 1905 of FIG. 19. In step 2002, the routine receives the two experimental results Eₓ and E_{y}, and sets the local variable sum2 to 0. Then, in steps 2004-2007, the routine computes the four terms representing the overlap between Aₓ and E_{y}, Dₓ and E_{y}, A_{y} and Eₓ, and D_{y} and Eₓ, respectively, as discussed above, using one of the two interval-overlap metrics O_{i,j} and Oᵢ′, or another of the essentially limitless number of possible interval-overlap metrics. In an optional step 2008, the contents of sum2 may be normalized, as in the case when overlap metric O_{i,j} is used, as discussed above.

Although the present invention has been described in terms of particular embodiments, it is not intended that the invention be limited to this embodiment. Modifications within the spirit of the invention will be apparent to those skilled in the art. For example, an essentially limitless number of embodiments of the present invention can be obtained by implementing the method embodiments of the present invention using different programming languages, control structures, data structures, modularization, and other, common programming parameters. Method embodiments of the present invention may be encoded in firmware, software, or a combination of software and firmware and included in analytical instruments and data-analysis systems of various types. As discussed above, any of an essentially limitless number of different arithmetic comparisons may be used to compute alternative interval-overlap metrics such as interval-overlap metrics O_{i,j} and Oᵢ′, discussed above. The various different alternative embodiments of the interval-overlap metric need to produce a range of values that describe degrees of similarity between the signals for two intervals in each of two result sets. Although particular normalization steps are discussed above, an essentially limitless number of different normalizations may be carried out in order to compute pairwise overlap metrics O(E₁,E₂) and O(ε). While the method embodiments of the present invention are particularly suited to aCGH results, they may be additionally applied to other types of genome-comparative experimental results in which aberrant intervals are identified. System embodiments of the present invention include processors and software programs that carry out the above method embodiments. Implementations of the methods of the present inventions may be included in software packages designed for experimental data collection and analysis.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for computing a quality metric for a set of k experimental results {E₁, E₂, . . . , Eₖ} in which aberrant chromosome intervals are identified, the method comprising:
computing pairwise overlap metrics for each possible pair of experimental results {Eₓ, E_{y}} selected from the k experimental results {E₁, E₂, . . . , Eₖ}; and
summing the computed pairwise overlap metrics to produce a numerical quality metric.

2. The method of claim 1 wherein, following summing the computed pairwise overlap metrics to produce a sum, the sum is divided by a term to produce a normalized quality metric.

3. The method of claim 1 wherein computing a pairwise overlap metric for a pair of experimental results {Eₓ, E_{y}} further comprises:
setting a result to 0;
for each amplification interval in Eₓ, computing an interval-overlap metric with respect to E_{y} and adding the computed interval-overlap metric to the result;
for each deletion interval in Eₓ, computing an interval-overlap metric with respect to E_{y} and adding the computed interval-overlap metric to the result;
for each amplification interval in E_{y}, computing an interval-overlap metric with respect to Eₓ and adding the computed interval-overlap metric to the result;
for each deletion interval in E_{y}, computing an interval-overlap metric with respect to Eₓ and adding the computed interval-overlap metric to the result; and
returning the result as the computed pairwise overlap metric.

4. The method of claim 3 wherein computing an interval-overlap metric further comprises:
for an amplification interval i in a first experimental result,
computing an interval-overlap O_{i,j} with respect to each amplification interval j in a second experimental result; and
selecting as the computed interval-overlap metric the largest valued computed interval-overlap O_{i,j}.

5. The method of claim 4 wherein an interval-overlap O_{i,j} is computed as the length of overlap between intervals i and j divided by the sum of the lengths of intervals i and j.

6. The method of claim 3 wherein computing an interval-overlap metric further comprises:
for an deletion interval i in a first experimental result,
computing an interval-overlap O_{i,j} with respect to each deletion interval j in a second experimental result; and
selecting as the computed interval-overlap metric the largest valued computed interval-overlap O_{i,j}.

7. The method of claim 6 wherein an interval-overlap O_{i,j} is computed as the length of overlap between intervals i and j divided by the sum of the lengths of intervals i and j.

8. The method of claim 3 wherein computing an interval-overlap metric further comprises:
for an aberrant interval i in a first experimental result, computing the absolute value of the difference between a signal measured for interval i and a signal measured for a corresponding interval i in a second experimental result.

9. Computer instructions that implement the method of claim 1 encoded in a computer-readable medium.

10. A system for computing a quality metric for a set of k experimental results {E₁, E₂, . . . , Eₖ} in which aberrant chromosome intervals are identified comprising:
a processor; and
a computer program running on the processor that
computes pairwise overlap metrics for each possible pair of experimental results {Eₓ, E_{y}} selected from the k experimental results {E₁, E₂, Eₖ}; and
sums the computed pairwise overlap metrics to produce a numerical quality metric.

11. The system of claim 10 wherein, following summing the computed pairwise overlap metrics to produce a sum, the computer program divides the sum by a term to produce a normalized quality metric.

12. The system of claim 10 wherein the computer program computes a pairwise overlap metric for a pair of experimental results {Eₓ, E_{y}} by:
setting a result to 0;
for each amplification interval in Eₓ, computing an interval-overlap metric with respect to E_{y} and adding the computed interval-overlap metric to the result;
for each deletion interval in Eₓ, computing an interval-overlap metric with respect to E_{y} and adding the computed interval-overlap metric to the result;
for each amplification interval in E_{y}, computing an interval-overlap metric with respect to Eₓ and adding the computed interval-overlap metric to the result;
for each deletion interval in E_{y}, computing an interval-overlap metric with respect to Eₓ and adding the computed interval-overlap metric to the result; and
returning the result as the computed pairwise overlap metric.

13. The system of claim 12 wherein computing an interval-overlap metric further comprises:
for an amplification interval i in a first experimental result,
computing an interval-overlap O_{i,j} with respect to each amplification interval j in a second experimental result; and
selecting as the computed interval-overlap metric the largest valued computed interval-overlap O_{i,j}.

14. The system of claim 13 wherein an interval-overlap O_{i,j} is computed as the length of overlap between intervals i and j divided by the sum of the lengths of intervals i and j.

15. The system of claim 12 wherein computing an interval-overlap metric further comprises:
for an deletion interval i in a first experimental result,
computing an interval-overlap O_{i,j} with respect to each deletion interval j in a second experimental result; and
selecting as the computed interval-overlap metric the largest valued computed interval-overlap O_{i,j}.

16. The method of claim 15 wherein an interval-overlap O_{i,j} is computed as the length of overlap between intervals i and j divided by the sum of the lengths of intervals i and j.

17. The system of claim 12 wherein computing an interval-overlap metric further comprises:
for an aberrant interval i in a first experimental result, computing the absolute value of the difference between a signal measured for interval i and a signal measured for a corresponding interval ī in a second experimental result.
